(19) United States
(12) Patent Application Publication
(10) Pub. No.: US 2003/0219487 A1
(43) Pub. Date: Nov. 27, 2003

(54) Process for the preparation of a small molecule gel via the addition of a branching additive

(76) Inventors: Xiang Yang Liu, Singapore (SG); Prashant D. Sawant, Singapore (SG)

Correspondence Address: One World Trade Center, Suite 1600, 121 S.W. Salmon Street, Portland, OR, 97204, US

(21) Appl. No.: 10/376,645

(22) Filed: Feb. 26, 2003

Related U.S. Application Data
(60) Provisional application No. 60/359,998, filed on Feb. 28, 2002.

(51) Int. Cl. A61K 9/14

(52) U.S. Cl. 424/486

(57) ABSTRACT

This invention relates to a process for the preparation of a small molecule gel, the process comprising heating a mixture of a gelling agent and a solvent to dissolve the gelling agent in the solvent; cooling the mixture to form a gel; and adding a branching additive to the mixture either before, during or after the heating, but prior to cooling. The invention also relates to a gel comprising a small molecule gelling agent, a solvent and a branching additive.

### CROSS-REFERENCE TO RELATED APPLICATION

This application claims the benefit of U.S. Provisional Patent Application No. 60/359,998 filed Feb. 28, 2002, which is incorporated herein by reference.

### FIELD OF THE INVENTION

The present invention relates to a process for the preparation of an organogel from organic small molecule gelling agents, and to the organogel prepared by such a process.

### BACKGROUND OF THE INVENTION

The term “gel” has taken various definitions in the past, as the term has been used to refer to different materials that display a wide range of properties. Most gels, however, fall within the broad definition of “a continuous interconnecting network that envelops a continuous liquid phase”.

Traditionally, gels have been composed of crosslinked polymers having large molecular weights, the polymers and crosslinking agents being connected through covalent bonds. More recently, it has been found that the aggregation of low molecular weight compounds in organic solvents can result in the formation of organogels. These organogels consist of self-organised interconnecting three-dimensional interconnecting networks, which networks can immobilise liquids upon gel formation.

Discoveries in the field of gels formed by small molecule gelling agents have been mostly limited to the identification of suitable gelling agents and solvents, and in the application of the gels formed. Organogels obtained from small molecule gelling agents have been found to have important applications in various fields, such as coatings, lithography, catalyst supporters, food processing, scaffolds for tissue engineering, drug delivery, tailor-made functional materials, cosmetics, photography, novel separation and nanostructured materials engineering. Apart from this, organogels are also applied in fragrance delivery, inks, paints, smart and responsive gel and display media where polymeric systems are currently in use. These compounds have also been shown to have the potential to absorb the oil phase selectively from an oil-water mixture, and they can be utilised in accidental oil spillage in large bodies of water.

Identification of suitable gelling agents and identification of the solvents in which these gelling agents are able to perform the interlinkage required to form gels has been limited to random or semi-random experimentation. These experiments usually consist of placing a candidate compound, that has a low molecular weight, in various solvents and subjecting the mixture to a standard process for preparing small molecule gels. The standard process usually consists of heating the mixture to dissolve the candidate compound in the solvent, which is then followed by a natural cooling step to see if the low molecular weight compound displays any gel forming tendencies. The only predictability in the identification of small molecule gelling agents has resided in the fact that certain classes of compounds, having similar structural elements or chemical functionality, have been shown to display similar gel forming tendencies.

### SUMMARY OF THE INVENTION

This invention provides a process for the preparation of small molecule gel, the process comprising heating a mixture of a gelling agent and a solvent to dissolve the gelling agent in the solvent; cooling the mixture to form a gel; and adding a branching additive to the mixture either before, during or after the heating, but prior to cooling.

The invention also provides small molecule gels prepared through the process described above.

### DESCRIPTION OF THE FIGURES

FIG. 1 illustrates the process for the preparation of a gel-fibre network architecture with and without branching additives.

FIG. 2 displays a photograph showing the effect of branching additives (from left to right, the additives are galactose (comparative), Gantrez AN-139, xylose (comparative) and EVACP) on the transparency of an organogel (Lanosta-8,24-dien-3β-ol C₃₀H₅₀O (L-DHL), prepared from a lanosterol/dihydrolanosteral mixture in Di-isooctylphthalate (C₈H₁₇COO)₂C₆H₄ (DIOP) as solvent). The photograph was taken 3 hours after preparation of the gel.

FIG. 3 displays a strain (G′) analysis of the improved strength of organogel fibres when the organogel is prepared in the presence of EVACP, against applied strain.

FIG. 4 displays the X-ray analysis of the crystalinity of Lanosta-8,24-dien-3β-ol C₃₀H₅₀O (L-DHL) in powder form, the crystalinity of the same compound obtained from a di-isooctylphthalate (C₈H₁₇COO)₂C₆H₄ (DIOP) solution, and the crystalinity of a gel of the same compound prepared from DIOP, with the addition of EVACP as branching agent.

FIG. 5 displays SEM images of (a) separate fibres occurring in the 10 w/v % L-DHL/DIOP system, and (b) interconnected fibre network in 10 wt % L-DHL/DIOP system after adding 0.004 wt % EVACP as a branching agent. The system without branching additives gives rise to an opaque paste as shown in the right upper corner of (a), while the presence of branching additive gives rise to a transparent and tough gel as shown in the right upper corner of (b). The length of the bar in both (a) and (b) is 1 μm.

FIG. 6 displays the effects of EVACP concentration on the micro- or nanofibre structure and rheological properties of a L-DHL gel. The increase of EVACP concentration C_{EVACP} gives rise to the reduction of the mesh size of L-DHL network (cf. a and b) and the rise of G* (c). FIG. 6(a) displays a fibre network of L-DHL obtained from 10 w/v % L-DHL in DIOP with 0.01 w/v % EVACP, while FIG. 6(b) displays a fibre network of L-DHL obtained from 10 w/v % L-DHL in DIOP with 0.1 w/v % EVACP. FIG. 6(c) displays the dependence of G* on C_{EVACP} (10 wt % L-DHL in DIOP).

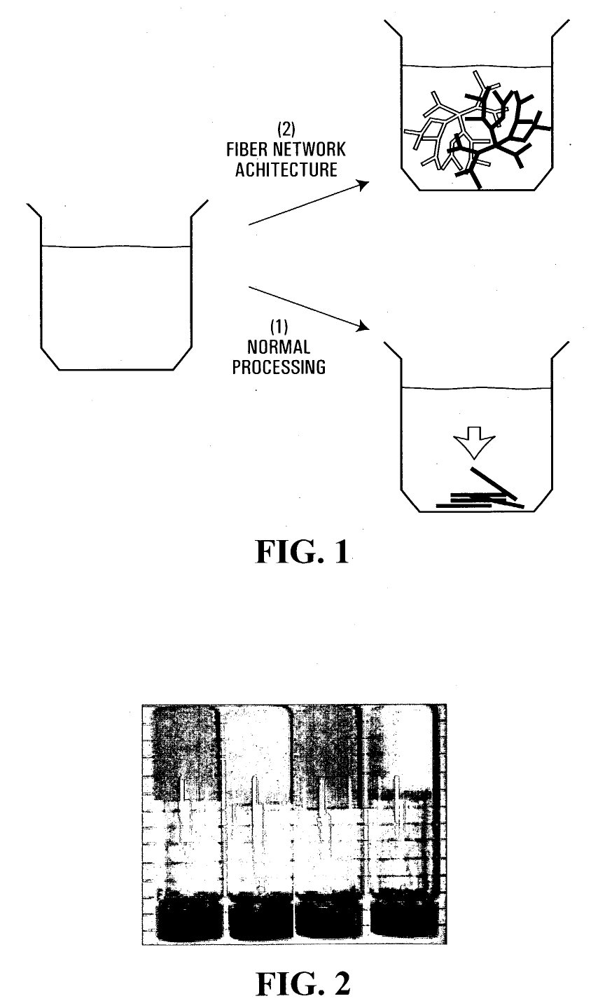

### DETAILED DESCRIPTION OF THE PREFERRED EMBODIMENTS

Gels (also referred to organogels, both terms being used interchangeably) prepared in accordance with the invention comprise arrays of molecules which are interconnected to each other via fibril branching, thus turning a liquid into a gel-like solid. The precise molecular arrangement of the molecules, and consequently the macroscopic qualities of the networks, are determined by the characteristics of the individual molecules and the nature of the solvent.

In one embodiment, a method for preparing gels from small molecule gelling agents is provided. This process can be applied, for example, to known poor gelling agent systems in order to obtain gels having superior macroscopic properties such as porosity, transparency, stability, hardness, and viscoelasticity.

Processes of the invention can also be applied to small molecule candidates that are unsuitable for producing gels when subjected to traditional small molecule gelling techniques, or that fail to produce gels when combined with specific solvents. When treated with traditional processes, these small molecule candidates are crystallised out of solution instead of forming clear gels, resulting in opaque. gels or pastes that have poor rheological properties and transparency. These poor properties are due to the fact that needle-like crystallites are formed. The introduction of a branching additive (also referred to as branching agent in this application, both term being used interchangeably) to the mixture comprising the small molecule candidate and the solvent can lead to three dimensional interconnecting network structures instead of crystalline needles, which has a beneficial effect on the macroscopic properties of the gel prepared.

Process

In one embodiment, a small molecule gelling agent is dissolved in a suitable solvent and the resulting solution is permitted to cool after a branching additive has been added to the solution. The additive is added before the cooling step of the process, either before, during, or after the heating step. It is preferred for the small molecule gelling agent to be completely dissolved, as a sparingly dissolved small molecule gelling agent may not give reproducible results as the nucleation and growth kinetics would be affected. It is not essential, however, that the small molecule gelling agent be completely dissolved in solvent.

The concentration of small molecule gelling agent in the mixture is preferably from about 0.1% w/v to about 20% w/v, more preferably from about 4% w/v to about 15% w/v. It is not essential, however, that the concentration of small molecule gelling agent fall within these ranges.

The concentration of branching agent in the mixture is preferably above 0.001% w/v, and preferably from about 0.001% w/v to about 0.1% w/v. It is not essential, however, that the concentration of the branching agent be within these ranges. The upper limit given in the above preferred range may not be applicable, as in some embodiments, increases in the concentration of the branching agent simply leads to thinner fibres and to smaller pore sizes in the gel. The concentration of the branching agent can thus be controlled to obtain gels having required porosity or mechanical properties.

The temperature to which the mixture is heated should be higher than the dissolution temperature of the gelling agent in the solvent, and less than the boiling point of the solvent. The solvent used in the preparation of a small molecule gel preferably has a boiling point which is fairly high, for example from about 170° C. to about 190° C. In some examples, the dissolution temperature of the small molecule gelling agent in the solvent is from about 80° C. to about 100° C., and the mixture is heated to 120° C. to ensure the fast and complete dissolution of the gelling agent.

Upon cooling, the mixture comprising the gelling agent, the solvent and the branching additive becomes supersaturated, at which point the gelling agent fibrils crystallise out of solution. The presence of the branching agent during the cooling step can modify the crystallisation of the gelling agent, leading to the branching of the fibrils to give interconnecting networks that form a gel. The mixture is cooled or allowed to cool to form a gel (e.g. cooled to about 30° C., 20° C., 10° C., 0° C. or −10° C.). The cooling of the mixture can be effected, for example, by removal of the mixture from the heating source and by permitting the mixture to rest at ambient temperature, e.g. room temperature. The cooling step can also be carried out, for example, by placing the mixture in a water bath, an ice bath or a refrigerator.

Gelling Agent

Suitable gelling agents include Small Molecule Gelling Agents (SMGA). These gelling agents differ from the polymeric materials traditionally used to form gels in that they have low molecular weights, usually of 3000 g/mol or less, and in some cases molecular weights of 1000 g/mol or less.

As mentioned above, SMGAs that have already been shown to give gels can be used, in this case to give gels that have improved characteristics. Known gelling agents have very varied structural and chemical elements, and examples of suitable SMGAs include, but are not limited to the following classes of molecules: fatty acid derivatives, steroid derivatives such as D-3,-hydroxy-17,17-dipropyl-17a-azahomoandrostanyl-17a-oxy (STNO) and D-3,-hydroxy-17,17-dipropyl-17a-azahomoandrostanyl-17a-aza (STHN), gelling agents containing steroidal and condensed aromatic rings, such as anthryl and anthraquinone appended steroid-based gelling agents, for example 2,3-Bis-n-decyloxyanthracene (DDOA) and 2,3-Bis-n-decyloxyanthraquinone (DDOA), azobenzene steroid-based gelling agents, such as molecules having a highly polar azobenzene group linked at C3 of a steroidal moiety, amino acid-type organogelators, and organometallic compounds, such as mononuclear copper β-diketonates. In some cases, two or more gelling agents can be used in combination to prepare the small molecule gels. Specific examples of the above classes of compounds and other known gelling agents can be found, for example, in Low Molecular Mass Gelators of Organic Liquids and the Properties of Their Gels, by Pierre Terech and Richard G. Weiss (Chem. Rev. 1997, 97, 3133-3159), the contents of which are hereby incorporated by reference.

Suitable solvents include, without limitation, n-hexane, n-heptane, n-octane, paraffin, cyclohexane, methylcyclohexane, decalin, carbon tetrachloride, carbon disulfide, benzene, toluene, p-xylene, nitrobenzene, m-cresol, 1,2-dichloroethane, dichloromethane, chloroform, diethyl ether, dipropyl ether, diphenyl ether, tetrahydrofuran, 1,4-dioxane, ethyl formate, methyl acetate, ethyl acetate, ethyl malonate, acetone, methyl ethyl ketone, N,N-dimethylformamide, N,N-dimethylacetamide, dimethyl sulfoxide, N-methyl-2-pyrrolidone, acetonitrile, methanol, ethanol, 1-propanol, 1-butanol, 1-octanol, benzyl alcohol, acetic acid, hexanoic acid, acetic anhydride, propyl amine, diethylamine, triethylamine, aniline, pyridine, triethylsilane, trimethylchlorosilane, diemthylpolysiloxane, cyclomethicone, trifluoroethanol, glycerol and water. Other suitable solvents can be found in Terech and Weiss (1997), which is identified above. As with the gelling agents, two or more solvents can be combined to prepare a small molecule gel.

Small molecule candidates that proved to be unsuitable for forming gels with traditional processes can also be used. Examples of small molecule gelling agent candidates which were unsuitable for forming gels with traditional processes and which can form gels with the processes of the present invention include lanosterol/dihydrolanosterol mixture, N-lauroyl-L-glutamic acid di-N-butylamide (LBADB), cholesteryl 4-(2-anthryloxy) butanoate, and methyltrioctadecylammonium iodide. Numerous solvents are suitable for use with these gelling agents, for example di-isooctylphthalate (DIOP), isostearyl alcohol, 2-butane-1,4 diol, 7-tridecanone, 2-octanone, 2-undecanone, 4-heptanone, 1,2-dimethoxyethane, 1-pentanol, acetonitrile, and 1-propanol.

Branching Additive

The gel-forming process is changed by the addition of branching additives. Without being bound by theory, it is believed that the formation of the 3D network of the gel in the presence of the branching agent takes place via a “non-crystallographic” branching mechanism. In this mechanism, the branching additive deposits on the tip of a fibre of the gelling agent as it is formed, thus creating two daughter fibres at the tip. This branching mechanism happens repeatedly, and results in the formation of a three-dimensional interconnected fibre network, which forms the pores of the gel.

To promote the branching of the fibres, the branching additive should be adsorbed effectively on the growing fibres to disrupt and to hinder the normal growth in the axial orientation. While the selection of a suitable branching additive is dependent on the nature of the gelling agent and of the solvent used, certain broad principles can be used to aid in the selection of a suitable branching agent. It is preferable that the branching agent (a) be a large molecule, for example a polymer, (b) have a rigid structure, (c) have structural units complimentary to the gelling agent, and (d) be able to interrupt the growth of the crystal layers along the fibre surface.

It is preferable that the branching agent be a large, rigid molecule because long and relatively rigid molecules have a much higher surface activity, thus adsorbing more strongly to the crystal surface. Large molecules are more strongly adsorbed to the crystal surface because, as they adsorb to the surface, they displace solvent molecules which are semi-bonded to the crystal surface. The larger the branching agent used, the more solvent molecules are displaced and the larger the entropy gain in the system, which leads to a more stable adsorption. Therefore, larger or longer molecules, such as polymers or macromolecules, will result in a larger entropy increase when they are adsorbed onto the crystal surface.

Adsorption (which in this context also encompasses absorption and chemisorption) of the branching agent onto the crystal surface, however, gives rise a separate competing phenomenon, which leads to a loss of entropy. Absorbance of a large molecule to the crystal surface leads to a loss of entropy because such large molecules relinquish their ability to change conformation when they are adsorbed, which introduces order in the system. It is for this reason that rigid branching agents are preferred, as such molecules do not give rise to a large loss of entropy upon adsorption, as they do not have a great range of possible conformational change prior to adsorption. As such, large and rigid molecules are more apt to provide an overall gain in entropy upon adsorbing to the crystal surface, thus providing to a more stable attachment. The rigidity of the branching agent can be controlled in various ways, for example by using a molecule that has intramolecular bonds (e.g. hydrogen bonds, double or triple covalent bonds, or rigid rings) in its backbone. Rigidity can also be increased by the presence of bulky functional groups in the branching agent molecule, as these groups deter rotation of the molecule, and they can stabilise the molecule through steric repulsion. Similarly, polyelectrolyte branching agents can display improved rigidity as the charged groups avoid each other due to electric repulsion, which hinders chain rotation.

The branching additive can also have functional groups that are apt to interact with the crystal surface, thus leading to stronger adsorption. The nature of these functional groups can vary for different gelling agents, but smaller and more flexible functional groups can prove to be desirable since the surface of the crystal is usually highly ordered and stiff, and smaller functional groups can better adjust their positions to obtain maximal interaction the surface. The interaction between the branching agent and the crystal surface can be achieved, for example, via hydrogen bonding, covalent bonding, ionic bonding, or van der waals forces.

It is also important that the branching agent be able to interrupt the crystal growth. This interruption can be due to repulsion effects, such as those originated by steric, electrostatic, polar/nonpolar or hydrophilic/hydrophobic forces, and these repulsion effects can be achieved by functional groups that are attached to the backbone of the branching additives.

In some embodiments, it is also preferable for the branching agent to have a fairly low solubility.

Suitable branching additives include, for example, ethylvinyl/ethylvinyl acetate copolymer (EVACP)[approximate molecular weight of 100,000], which is available from SP² Scientific Polymer Products Inc, and poly(methyl vinyl ether)/maleic anhydride copolymer (I) [approximate molecular weight of 1,080,000], which is commercially available as Gantrez AN-139, from ISP Europe.

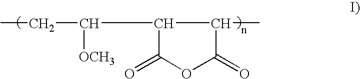

FIG. 2 displays the effect of branching additives on the transparency of an organogel (Lanosta-8,24-dien-3β-ol C₃₀H₅₀O (L-DHL) in Di-isooctylphthalate (C₈H₁₇COO)₂C₆H₄ (DIOP)). Each assay was treated with a different additive, which are, from left to right, galactose, gantrez AN-139, xylose and EVACP. The saccharides (galactose and xylose) are clearly inferior to Gantrez and to EVACP, which conforms to the guideline that large, polymeric molecules are superior as branching agents.

Organogels

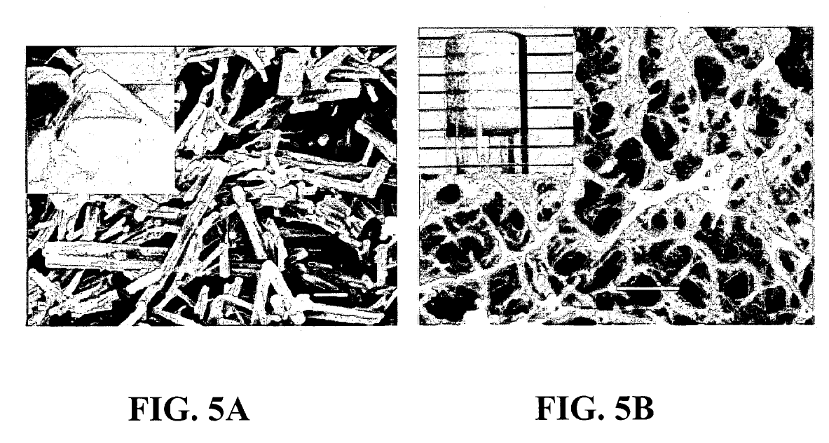

A process in accordance with the present invention can provide organogels that have enhanced properties. In general, the aforementioned process achieves (1) thinner fibres, (2) the formation of 3D interconnecting network structure instead of separate needle-like crystals from the same gelling agent/solvent system, and (3) the modification the mesh size of 3D interconnecting network structure. The pores in the 3D-network structure of the gels permit the immobilisation of liquids, thus improving liquid carrying capacity of the gels. Also improved are the transparency, elasticity, hardness and the capability of selectively carrying a specific compound (such as nanoparticles, proteins, drugs, chemicals and cells) of the gels. The effect of the branching agent can be seen in FIGS. 5(a) and 5(b), where a small molecule gelling agent (lanosterol/dihydrolanosterol mixture) is combined with a solvent (di-isooctylphthalate) and subjected to a traditional gellation process (FIG. 5(a)), and to a gellation process in accordance with the present invention (FIG. 5(b)) where a branching agent (EVACP) is used. The system where a branching agent is not used provides, upon cooling, a crystalline needle product that gives rise to an opaque gel as shown in the picture on the right upper corner of FIG. 5(a). FIG. 5(b), however, shows an interconnected fibre network that is obtained due to the presence of the branching agent. This system gives rise to a clear and tough gel of lanosta-8,24-dien-3β-ol C₃₀H₅₀O (L-DHL), as shown in the picture on the upper right corner of FIG. 5(b).

Additive components that do not affect gelation can also be introduced to the gel during the processes, to change certain characteristics of the gel, such as coloration.

The gels prepared from SMGAs usually differ from the gels that comprise crosslinked polymers, as they are held together by non-covalent forces, such as hydrogen bonding, van der waals forces, π-π interactions and ionic bonding. Since all these forces are reversible in nature, the gels prepared are usually themselves thermo-reversible.

In some embodiments, the porosity of the gels is 500 nm or less., while in other embodiments the porosity is in the range of from about 50 nm to about 500 nm, from about 200 nm to about 500 nm, or from about 50 nm to about 300 nm. It is not essential, however, that the gels have porosity within these ranges.

Since the interconnected fibre networks of the invention benefit from the presence of thinner fibres and to a more open network instead of needle like crystal formations, and that these thinner fibres are less apt to diffract light than their needle counterparts, the quality of the fibre networks can be determined by their transparency. While transparency of the gel is not essential, a high level of transparency is preferred as it indicates the presence of smaller fibres. Transparency of the networks can be determined roughly by eye, or a more thorough evaluation can be carried out by measuring the absorbency of the gels at various wavelengths of light. The average absorbency of a compound over a range of wavelengths, for example from 500 to 600 nm, gives a good measure of the transparency of the fibre network. The stability of the organogels can also usually be verified visually, and in some embodiments, use of branching additives to enhance stability of gels can cause increases in stability of from about 2 to about 6 hours.

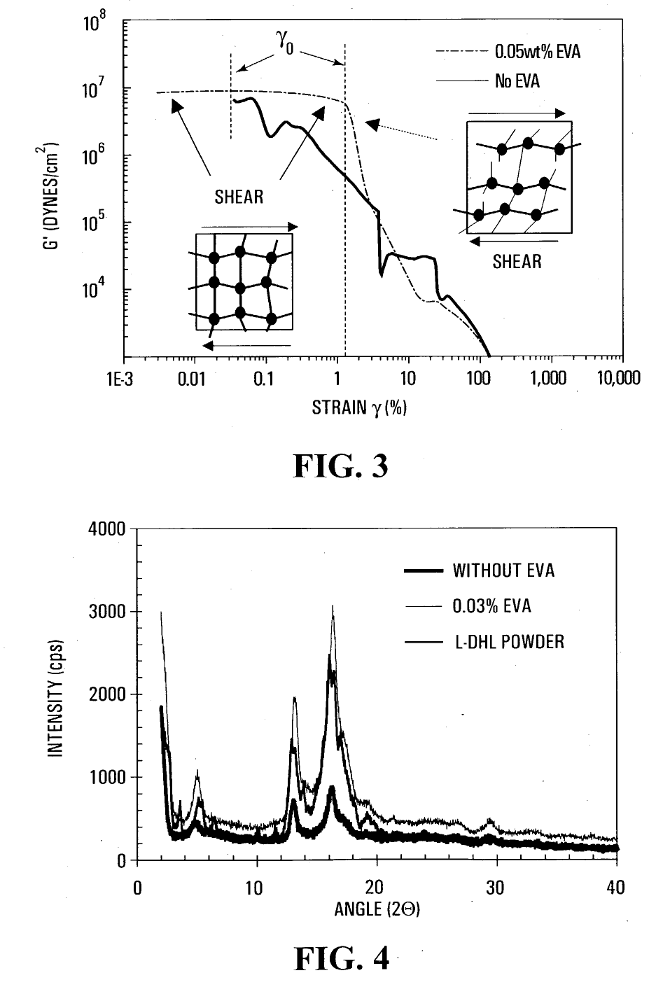

The interconnecting fibre network obtained with the branching additive significantly changes the elastic and the viscous properties of the gel. Viscoelasticity can be used to assess the quality of the gels prepared, and this characteristic can be quantitatively measured as the “limit of linearity, γₒ”. The value for γₒ is calculated by applying different forces to the gel, and by measuring the elastic modulus (G*) or the storage modulus (G′) values obtained. The relationship between the G* and G′ is given by the equation G*=G′+iG″, where G′ describes the elastic qualities of the substance and G″ (loss modulus) describes the viscosity qualities of the substance. The elastic modulus is a measure of overall resistance to deformation. The strain applied affects the 3D interconnecting microstructure of the gel, and at a specific level of strain, which is defined as γₒ, the mesh structures begins to break down, which is seen by a decrease of the elastic modulus. Until this value of γₒ is attained, the elastic modulus is unchanged and it usually provides a linear response that is parallel to the X-axis, as the strain is increased. A higher value of γₒ indicates that the microstructures in the gel are more resistant to deformation when strain is applied, which is usually due to the higher number of discrete fibres in the gel. A high value for γₒ is usually indicative of a better gel. In some embodiments, a value of about 0.1% for γₒ is observed for gels prepared without branching additives, while γₒ values of about 1% are observed for gels prepared with a branching additives. A comparison of G′ values with varying levels of strain for gels prepared with or without a branching additive is found in FIG. 3. It can be seen in the Figure that a gel prepared with branching additives (0.05% wt EVACP) is more resilient than a similar gel prepared without branching additives.

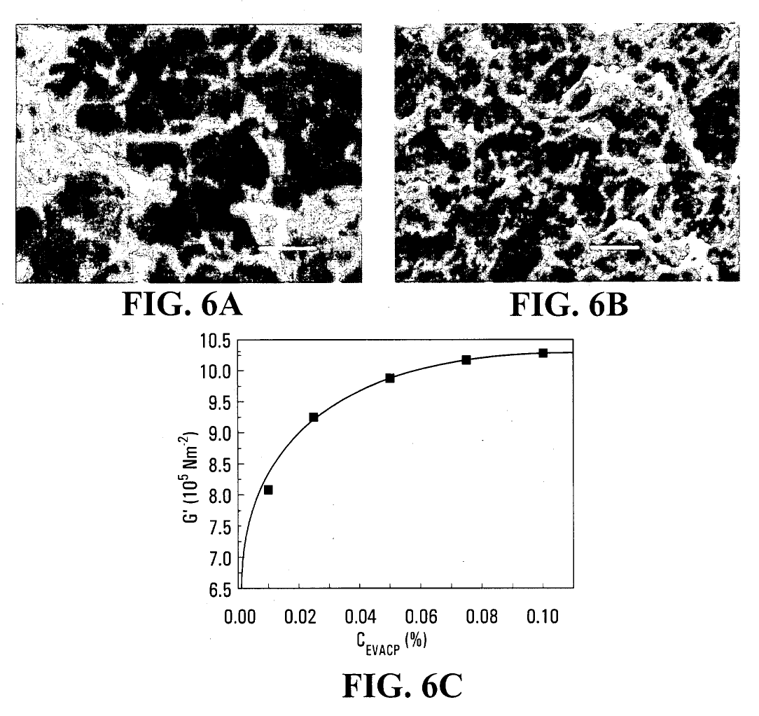

In addition to the nature of the gelling agent and the solvent, the concentration of the branching agent also has an effect on the characteristics of the gel. FIG. 6 displays the effects of the concentration of a branching agent (EVACP) on the fibre structure (FIGS. 6a and 6b) and on the rheological properties (FIG. 6c) of L-DHL gel. An increase of EVACP concentration, C_{EVACP}, gives rise to the reduction of the mesh size of L-DHL network (FIGS. 6a and 6b) and the rise of the complex modulus, G* (FIG. 6c). FIG. 6a shows the formation of the fiber network of L-DHL obtained from 10% w/v L-DHL in DIOP in presence of 0.01% w/v of EVACP. FIG. 6b shows the fiber network of L-DHL obtained from 10% w/v of L-DHL in DIOP in presence of ten fold increase in the concentration of EVACP (0.1% w/v). The Figures show that a higher concentration of EVACP enhances the branching of the fibres in the gel, and that it can affect the viscoelastic properties of the gel.

In some embodiments, it has been observed that introduction of a branching agent to the gellation process leads to higher quality gels, while leaving the overall crystalinity of the gel substantially unaltered. X-ray diffraction studies of the gels with or without branching agents, as seen in FIG. 4, show that the peaks measured are very similar whether or not the additive is present. In FIG. 4, the X-ray diffraction peaks for L-DHL are almost identical when EVACP is present, when EVACP is not present, and for L-DHL powder obtained from a xerogel. The xerogel of L-DHL is obtained from the unstable gel by using supercritical CO₂ extraction methods. Therefore, while the micro- or nanostructure of the fibres is changed with the addition of a branching agent, in some embodiments the crystalinity of the fibres substantially remains the same.

Uses

In addition to the fields of use discussed earlier, gels prepared with a process according to the present invention can be used to prepare devices capable of cell detection, isolation, and genetic analysis. Such devices can provide diagnostic, therapeutic, and prognostic information. For the isolation devices, which can be used for cell isolation, channels in the gel vary in gap width across the device (e.g. 20, 15, 10, and 5 μm). Devices were fabricated with three different channel depths (20, 10, and 5 μm), and any one device has the same depth throughout the array. Mixing the cell mixtures with Pluronics PF-108 (tri-block co-polymer) generates a hydrophilic surface for flow and prevents cell adhesion to the gel. Gels can be capable of fractionating and isolating cell types of interest from a complex heterogeneous starting mixture, like whole blood.

The following examples are offered by way of illustration and not by way of limitation.

### EXAMPLES

### Example 1 (Comparative)

0.1 g of lanosterol/dihydrolanosterol (as gelling agent) in a mass ratio of 56:44 was added to 10 ml of di-isooctylphthalate (DIOP) in 15 ml bottles. The gelling agent was dissolved in DIOP by heating in the oven at 120° C. for one hour until the L-DHL had dissolved completely in the organic solvent to form a clear transparent solution. The hot solution was then cooled to approximately 20° C. by natural cooling. FIG. 5(a) displays the ESEM image of a naturally cooled microstructure without additives. The ESEM image shows that the lack of a branching additive in the process renders the small molecule gelling agent incapable of forming a gel. FIG. 3 displays a graphical analysis of the elastic modulus (G′) versus strain for the naturally cooled microstructure made without a branching additive.

### Example 2

In a process similar to that disclosed in Example 1, 0.03% w/v of an ethylvinyl acetate/vinyl acetate co-polymer (EVACP) having a co-monomer mass ratio of 60:40, was added before the heating process. FIG. 2 displays the effect on transparency produced by the presence of additives in the reaction, the right-most vial displaying the transparency of the three-dimensional interconnected fibre network when EVACP is used as an additive. FIG. 3 displays the change in elastic modulus as strain is applied to the gel formed with the branching additive. FIG. 4 displays the X-ray diffraction pattern of the L-DHL gel, when it is prepared with EVACP as a branching additive. FIG. 5(b) displays. the ESEM image of the three dimensional structure formed in the L-DHL gel prepared in the present example.

The example above demonstrates that the use of a branching additive can lead to the production of a gel from a gelling agent that was unable to form a gel from traditional small molecule gellation processes. Table 1 provides additional examples of small molecule gelling agents that can form gels when subjected to a process according to the present invention, while forming crystalline needles or pastes under traditional processes. Table 1 also provides example of solvents that are suitable for preparing gels with the identified small molecule gelling agents. Examples of suitable branching agents for use with the combinations of gelling agents and solvent identified in Table 1 include EVACP and Gantrez AN-139.

TABLE 1
Combinations of Small Molecule Gelling Agents and Solvents

| Small Molecule Gelling Agent | Solvent |
|---|---|
| Cholesteryl | 2-Butane-1,4diol |
| 4-(2-anthryloxy) | 7-Tridecanone |
| butanoate (CAB) | 2-Octanone |
| | 2-Undecanone |
| | 1,2-Dimethoxyethane |
| Methyltrioctadecylammonium | 1-Pentanol |
| iodide | Cyclohexane |
| | Acetonitrile |
| | 1-Propanol |
| N-lauroyl-L-glutamic acid | Isostearyl Alcohol |
| di-N-butylamide | |

All publications, patents and patent applications cited in this specification are herein incorporated by reference as if each individual publication, patent or patent application were specifically and individually indicated to be incorporated by reference. The citation of any publication is for its disclosure prior to the filing date and should not be construed as an admission that the present invention is not entitled to antedate such publication by virtue of prior invention.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A process for the preparation of a small molecule gel, the process comprising heating a mixture of a gelling agent and a solvent to dissolve the gelling agent in the solvent; cooling the mixture to form a gel; and adding a branching additive to the mixture either before, during or after the heating, but prior to cooling.

2. The process according to claim 1, wherein the gelling agent is has a molecular weight of 3000 g/mol or less.

3. The process according to claim 1, wherein the gelling agent is has a molecular weight of 1000 g/mol or less.

4. The process according to claim 1, wherein the gelling agent is selected from the group consisting of lanosterol/dihydrolanosterol (L-DHL), N-lauroyl-L-glutamic acid di-N-butylamide (LBADB), cholesteryl 4-(2-anthryloxy) butanoate and methyltrioctadecylammonium iodide.

5. The process according to claim 1, wherein the gelling agent is present in a concentration of from about 0.1% w/v to about 20% w/v.

6. The process according to claim 1, wherein the gelling agent is present in a concentration of from about 4% w/v to about 15% w/v

7. The process according to claim 1, wherein the solvent is selected from the group consisting of di-isooctylphthalate (DIOP), 2-butane-1,4 diol, 7-tridecanone, 2-octanone, 2-undecanone, 4-heptanone, 1,2-dimethoxyethane, 1-pentanol, acetonitrile, and 1-propanol.

8. The process according to claim 1, wherein the branching additive is a polymer.

9. The process according to claim 1, wherein the branching additive is selected from the group consisting of ethylvinyl/ethylvinyl acetate copolymer, and poly-(methyl vinyl ether)/maleic anhydride copolymer.

10. The process according to claim 1, wherein the branching additive is present in a concentration above 0.001% w/v.

11. The process according to claim 1, wherein the branching additive is present in a concentration of from about 0.001% w/v to about 0.1% w/v.

12. A gel comprising a small molecule gelling agent, a solvent and a branching additive.

13. The gel according to claim 12, wherein the gelling agent is lanosterol/dihydrolanosterol, the solvent is di-isooctylphthalate, and the branching additive is ethylvinyl/ethylvinyl acetate copolymer or poly(methyl vinyl ether)/maleic anhydride copolymer.

14. The gel according to claim 12, wherein the gelling agent is N-lauroyl-L-glutamic acid di-N-butylamide, the solvent is isostearyl alcohol, and the branching additive is ethylvinyl/ethylvinyl acetate copolymer or poly(methyl vinyl ether)/maleic anhydride copolymer.

15. The gel according to claim 12, wherein the gelling agent is cholesteryl 4-(2-anthryloxy) butanoate (CAB), the solvent is selected from the group consisting of 2-butane-1,4diol, 7-tridecanone, 2-octanone, 2-undecanone, and 1,2-dimethoxyethane, and the branching additive is ethylvinyl/ethylvinyl acetate copolymer or poly(methyl vinyl ether)/maleic anhydride copolymer.

16. The gel according to claim 12, wherein the gelling agent is methyltrioctadecylammonium iodide, the solvent is selected from the group consisting of 1-pentanol, cyclohexane, acetonitrile and 1-propanol, and the branching additive is ethylvinyl/ethylvinyl acetate copolymer or poly(methyl vinyl ether)/maleic anhydride copolymer.
